(19) United States
(12) Patent Application Publication
(10) Pub. No.: US 2007/0213568 A1
(43) Pub. Date: Sep. 13, 2007

(54) Process for preparing relatively long-chain polyalkylene glycol diethers

(75) Inventors: Achim Stankowiak, Altoetting (DE); Alexander Snell, Altoetting (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 11/716,497

(22) Filed: Mar. 9, 2007

(30) Foreign Application Priority Data
Mar. 9, 2006 (DE) 10 2006 010 940.6

(51) Int. Cl. C07C 41/03

(52) U.S. Cl. 568/679

(57) ABSTRACT

The invention provides a process for preparing alkylene glycol diethers of the formula (I)

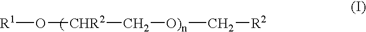

by converting compounds of the formula (II)

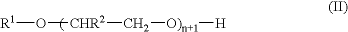

in which R¹ is hydrogen or C₁— to C₃-alkyl, R² is hydrogen, CH₃ or CH₂—CH₃ and n is from 5 to 500 in the liquid phase at temperatures between 170 and 300° C. in the presence of a Raney nickel catalyst which, based on the total weight of the catalyst, contains from 0.1 to 10% by weight of one or more other metals selected from transition groups I, VI and VII of the Periodic Table of the Elements.

The present invention relates to a process for preparing catenated alkylene glycol diethers having a molecular weight of at least 250 g/mol.

Alkylene glycol diethers have been used for some time as polar, aprotic, inert solvents. High molecular weight alkylene glycol diethers find use in particular in electrochemistry, as high-boiling solvents and as linear crown ethers in phase transfer catalysis.

For their preparation, so-called indirect processes, for example the Williamson Ether synthesis (K. Weissermel, H. J. Arpe “Industrielle Organische Chemie” [Industrial Organic Chemistry], 1998, page 179) or the hydrogenation of diglycol ether formal (DE-A 24 34 057), are employed industrially or described. However, both processes have disadvantages: the two-stage Williamson ether synthesis is of low economic viability as a result of the stoichiometric chlorine and alkali consumption, and the removal of the water of reaction and sodium chloride formed. The hydrogenation of formal is performed under high pressure, which requires high capital costs in the plant construction and is therefore unsuitable for relatively small production volumes.

In the so-called direct processes, alkylene oxide is inserted into a catenated ether in the presence of Lewis acids such as BF₃ (U.S. Pat. No. 4,146,736 and DE-A 26 40 505 in conjunction with DE-A 31 28 962) or SnCl₄ (DE-A 30 25 434). The disadvantage of these processes is that relatively large amounts of cyclic by-products, for example dioxane or dioxolane, are unavoidably formed. Moreover, these processes cannot be applied to relatively long-chain polyalkylene glycol ethers (high proportion of by-products).

An alternative synthesis means is the catalytic deformylation of glycols and methyl glycols:

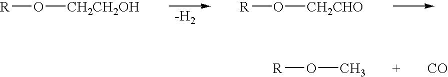

The patent DE 2 900 279 gives the first description of this synthesis route by the reaction of polyethylene glycols or polyethylene glycol monomethyl ethers in the gas phase at 250-500° C. in the presence of supported palladium, platinum, rhodium, ruthenium or iridium catalysts and hydrogen. The Japanese patent JP 60028429 describes the reaction of C₄ and longer-chain monoalkyl ethers using a nickel/rhenium catalyst supported on γ-alumina. In this process too, hydrogen is supplied continuously. Likewise known is the hydrogenation of secondary hydroxyl groups with hydrogen at standard pressure using supported nickel catalysts

(DE-A 38 02 783). In this process, the synthesis explicitly does not succeed when Raney nickel is used.

It is known from the patent U.S. Pat. No. 3,428,692 that heating of C₆— to C₁₂-chain monoalkyl and monophenyl ethers to 200-300° C. in the presence of nickel and cobalt catalysts allows the corresponding deformylated methyl-capped ethoxylates to be prepared. However, this forms mixtures of the desired methyl ethers with incompletely converted ethoxylates and 20-30% of unidentified aldehyde compounds.

EP 0 043 420 describes a similar process using palladium, platinum or rhodium catalysts, supported on Al₂O₃ or SiO₂.

All processes described in the current prior art are either of low selectivity or else technically very complicated and therefore economically unviable for the preparation of relatively long-chain alkylene glycol diethers. The object arising therefrom is achieved in accordance with the invention according to the claim.

Surprisingly, relatively long-chain alkylene glycols and alkylene glycol monoethers can be converted to the desired alkylene glycol diethers in a simple slurry process by metal catalysis. The synthesis succeeds quantitatively (>99%) and without formation of by-products. After the reaction, the catalyst can be removed completely in a simple filtration step (<1 ppm of metal).

The invention thus, provides a process for preparing alkylene glycol diethers of the formula (I)

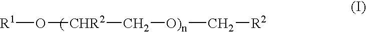

by converting compounds of the formula (II)

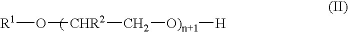

in which R¹ is hydrogen or C₁— to C₃-alkyl, R² is hydrogen, CH₃ or CH₂—CH₃ and n is from 5 to 500 in the liquid phase at temperatures between 170 and 300° C. in the presence of a Raney nickel catalyst which, based on the total metal content of the catalyst calculated as elemental metal, contains from 0.1 to 50% by weight of one or more other metals selected from transition groups I, VI and VIII of the Periodic Table of the Elements.

The conversion over the catalysts is effected preferably at from 200 to 250° C. The reaction is performed generally at standard pressure, but it is also possible to work under reduced or elevated pressure. The reaction time is generally between 4 and 10 hours.

R¹ is preferably H or methyl.

R² is preferably hydrogen.

n is preferably from 15 to 300.

The Raney nickel catalyst contains preferably from 0.2 to 25% by weight, in particular from 0.5 to 10% by weight, of one or more other metals selected from transition groups I, VI and VIII of the Periodic Table of the Elements. Preferred metals from transition groups I, VI and VIII of the Periodic Table of the Elements are palladium, iron, molybdenum, copper, chromium, cobalt, platinum, rhodium, ruthenium and iridium. These metals may either be doped on the same support material with the Raney nickel, or be added to the catalyst on a separate support material. In the case of the mixture of catalysts on separate supports, the term catalyst means the mixture. The metal content of the catalyst is always reported in percent of the total metal content. The total metal weight of the catalyst calculated as the elemental metal always corresponds to 100% by weight.

The process according to the invention will now be illustrated in detail using some examples:

### EXAMPLE 1

Synthesis of polyglycol dimethyl ether having a molar mass of approx. 500 g/mol

In a 250 ml three-neck flask, 361.7 g of polyglycol monomethyl ether (molar mass approx. 500 g/mol), 12.3 g of palladium on activated carbon (palladium content 0.6 g) and 19.4 g of anhydrous Raney nickel (nickel content 9.7 g) are stirred vigorously at 230° C. under protective gas. After 8 hours of reaction time, the reaction mixture is filtered at 80° C. through silica gel. The conversion is 98.6%. In the product, no nickel (AAS) or palladium (ICPOES) can be detected.

### EXAMPLE 2

Synthesis of polyglycol dimethyl ether having a molar mass of approx. 2000 g/mol

In a 250 ml three-neck flask, 399.5 g of polyglycol monomethyl ether (molar mass approx. 2000 g/mol), 19.7 g of palladium on activated carbon (palladium content 0.98 g) and 31.0 g of anhydrous Raney nickel (nickel content 15.5 g) are stirred vigorously at 230° C. under protective gas. After 6 hours of reaction time, the reaction mixture is filtered at 80° C. through silica gel. The conversion is 99.3%. In the product, no nickel or palladium can be detected.

### EXAMPLE 3

Synthesis of polyglycol dimethyl ether having a molar mass of approx. 4000 g/mol

In a 250 ml three-neck flask, 395.5 g of polyglycol monomethyl ether (molar mass approx. 4000 g/mol), 19.4 g of palladium on activated carbon (palladium content 0.97 g) and 30.6 g of anhydrous Raney nickel (nickel content 15.3 g) are stirred vigorously at 230° C. under protective gas. After 8 hours of reaction time, the reaction mixture is filtered at 80° C. through silica gel. The conversion is 98.8%.

### EXAMPLE 4 (COMPARATIVE)

Synthesis of polyglycol dimethyl ether having a molar mass of approx. 10 000 glmol

In a 250 ml three-neck flask, 331.5 g of polyglycol monomethyl ether (molar mass approx. 10 000 g/mol) and 18.7 g of anhydrous Raney nickel (nickel content 9.4 g) are stirred vigorously at 200° C. under protective gas. After 8 hours of reaction time, the reaction mixture is filtered at 80° C. through silica gel. The conversion is 86.1%.

### EXAMPLE 5

Synthesis of polyglycol dimethyl ether having a molar mass of approx. 10 000 g/mol

In a 250 ml three-neck flask, 332.4 g of polyglycol monomethyl ether (molar mass approx. 10 000 g/mol), 11.6 g of palladium on activated carbon (palladium content 0.58 g) and 18.3 g of anhydrous Raney nickel (nickel content 9.2 g) are stirred vigorously at 230° C. under protective gas. After 8 hours of reaction time, the reaction mixture is filtered through silica gel. The conversion is 99.0%.

### EXAMPLE 6

Synthesis of polyglycol dimethyl ether having a molar mass of approx. 10 000 g/mol

Analogously to Example 5, 332.4 g of polyglycol monomethyl ether (molar mass approx. 10 000 g/mol), 18.3 g of Raney copper (copper content 9.2 g) and 18.3 g of Raney nickel (nickel content 9.2 g) are stirred vigorously at 200° C. After 8 hours of reaction time, the reaction mixture is filtered through silica gel. The conversion is 89.2%.

### EXAMPLE 7

Synthesis of polyglycol dimethyl ether having a molar mass of approx. 10 000 g/mol

Analogously to Example 5, 332.0 g of polyglycol monomethyl ether (molar mass approx. 10 000 g/mol) and 18.2 g of Raney nickel (nickel content 9.1 g) doped with 3% by weight of chromium and 3% by weight of iron (based on the total weight of metals in the catalyst) are stirred vigorously at 220° C. After 8 hours of reaction time, the reaction mixture is filtered through silica gel. The conversion is 89.2%.

### EXAMPLE 8

Synthesis of polyglycol dimethyl ether having a molar mass of approx. 10 000 g/mol

Analogously to Example 5, 330.1 g of polyglycol monomethyl ether (molar mass approx. 10 000 g/mol) and 18.0 g of Raney nickel (nickel content 9.0 g) doped with 8% by weight of copper and 3% by weight of molybdenum (based on the total weight of metals in the catalyst) are stirred vigorously at 220° C. After 8 hours of reaction time, the reaction mixture is filtered through silica gel. The conversion is 93.4%.

1. A process for preparing alkylene glycol diethers of the formula (I) 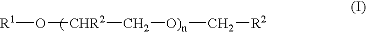
by converting compounds of the formula (II) 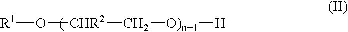
in which R¹ is hydrogen or C₁— to C₃-alkyl, R² is hydrogen, CH₃ or CH₂—CH₃ and n is from 5 to 500 in the liquid phase at temperatures between 170 and 300° C. in the presence of a Raney nickel catalyst which, based on the total weight of the catalyst, contains from 0.1 to 50% by weight of one or more other metals selected from transition groups I, VI and VIII of the Periodic Table of the Elements.

2. The process as claimed in claim 1, in which R¹ is H or methyl.

3. The process as claimed in claim 1, in which R² is hydrogen.

4. The process of claim 1, in which n is from 15 to 300.

5. The process of claim 1, in which the Raney nickel catalyst contains from 0.2 to 25% by weight of one or more other metals selected from transition groups I, VI and VIII of the Periodic Table of the Elements.

6. The process of claim 1, in which the metals from transition groups I, VI and VIII of the Periodic Table of the Elements are selected from the group consisting of palladium, iron, molybdenum, copper, chromium, cobalt, platinum, rhodium, ruthenium, iridium, and mixtures thereof.

7. The process of claim 1, in which the metals from transition groups I, VI and VIII of the Periodic Table of the Elements are doped with the Raney nickel on the same support material, or are added to the catalyst on a separate support material.
